(19) United States
(12) Patent Application Publication
(10) Pub. No.: US 2008/0194921 A1
(43) Pub. Date: Aug. 14, 2008

(54) Methods and systems for automatically evaluating data from at least one clinical study

(76) Inventors: Markus Schmidt, Nuremberg (DE); Siegfried Schneider, Erlangen (DE); Gudrun Zahlmann, Neumarkt (DE)

Correspondence Address: HARNESS, DICKEY & PIERCE, P.L.C., P.O.BOX 8910, RESTON, VA, 20195, US

(21) Appl. No.: 11/806,336

(22) Filed: May 31, 2007

Related U.S. Application Data
(60) Provisional application No. 60/900,344, filed on Feb. 9, 2007.

(51) Int. Cl. A61B 5/00

(52) U.S. Cl. 600/300

(57) ABSTRACT

In a method for automatically evaluating data from at least one clinical study, data may be assessed at a study management system based on at least one management criteria. At least one measured variable may be determined based on the assessed data, and at least one action correlated to at least one facility may be initiated based on the at least one measured variable. The at least one management criteria may be associated with the at least one clinical study.

### PRIORITY STATEMENT

This non-provisional U.S. patent application claims priority under 35 U.S.C. §119(e) to U.S. provisional patent application Ser. No. 60/900,344, filed on Feb. 9, 2007, the entire contents of which is incorporated herein by reference.

### BACKGROUND

Design, implementation and evaluation of clinical studies are extremely complex ventures involving a large number of people and institutions. Such ventures produce a large volume of data. The complexity of administration for this is immense. Conventionally, automatic study management systems are used as central support systems for design, implementation and evaluation of clinical studies.

In many cases, clinical studies are implemented in an image-based mode. In an image-based mode, image material (e.g., X-ray pictures of patients) is input into the clinical study directly, not merely as information extracted from the image data (e.g., in textual or numerical form).

A conventional study management system receives image data from external stations. The external stations are normally medical facilities, such as institutions, institutes, clinics, doctors' practices, etc., assigned the tasks of implementing the clinical study and creating the relevant image data.

A conventional automatic study management system includes a quality control module as a first receiving station for incoming data. In this example, not only is the image data checked, but also corresponding additional information correlated to the data is tested. Such additional information is, for example, metadata associated with the image data. Metadata may be header data for the image data. Examples of metadata include the time at which an image is taken, picture parameters for the image, role of the personnel tasked with taking or capturing the image, etc.

Conventionally, clinical studies do not have automatic study management systems. Such implementations involve manual evaluation of data supplied from a facility to the study. Thus, the volume of data handled is limited by available personnel resources. In addition, conventional study management systems do not use image data to make such assessments.

For example, a conventional clinical study involves the transmission of only a few images (e.g., the best or certain selected images) without any additional information to the central station tasked with the clinical study.

### SUMMARY

Example embodiments relate to methods and systems for automatically evaluating data associated with at least one clinical study. The data may be supplied to a data input module in an automatic clinical study data evaluation system from at least one facility performing the at least one clinical study.

Methods and systems according to at least some example embodiments provide a method for evaluating data from at least one clinical study supplied to a clinical study data evaluation system.

At least one example embodiment provides a method for automatically evaluating data from at least one clinical study. The data may be supplied to a data input module in an automatic study management system. For example, the data may be transmitted to the study management system by at least one facility performing the at least one clinical study. The data may be transferred from the data input module to a data management system. The data management system may further process the data. The study management system may assess the data according to the management criteria correlated to the clinical study. Using or based on this assessment, the study management system may determine at least one measured variable assessing the data for the management criteria. Based on the measured variable, the study management system may initiate at least one action correlated to the facility.

According to at least one example embodiment, the data or image data entering the study management system may be automatically assessed or analyzed and the corresponding information may be made available or utilized in performing at least one action. The action or actions may be automatically initiated according to rules of the study management system.

Using automatic study management systems according to example embodiments, a larger amount of data from the clinical study may be captured. For example, automatic study management systems according to example embodiments may capture all or substantially all data produced during one or more clinical studies. Additional information including, for example, Digital Imaging and Communications (DICOM) headers of magnetic resonance (MR) pictures (e.g., indicating how long the patient was in the MR system to produce the MR picture) may also be gathered and accompany the image data.

Example embodiments may allow evaluation of an increased amount of data. As a result, example embodiments may enable more specific and/or more detailed actions to be performed.

Example embodiments may be used to generate at least one measured variable relating to or associated with at least one facility during the course of one or more clinical studies by evaluating image data (e.g., images and/or metadata), determining results based on the measured variable, and performing one or more actions based on the determined results. The results may be determined in a given, specified or predefined manner based on one or more management criteria.

Data may be transmitted from the facility to the study management system via one or more (e.g., two different routes) data paths. For example, if the data input module is a standalone module, the data may be transmitted to the data input module, to the study management system and/or to the data management system.

Alternatively, the data input module may be part of or integrated with the data management system. In this example, the data may be transmitted directly or indirectly from the facility to the data management system. The study management system may access the data management system to fetch or obtain the data.

According to at least some example embodiments, data may be assessed in different ways, according to different management criteria. Management criteria may be, for example, study protocols, agreements with facilities relating to the clinical study, etc. This may allow the study management system to determine the study-related quality of the data transmitted from the facility. The study management system may also make inferences about the quality of the facility itself, and match an action more precisely to the quality of the relevant facility.

In another example, at least one measured variable may be determined using at least one management criteria such as a calculation rule correlated to the clinical study. For example the measured variable may be the number of patients examined and/or treated by the facility in the course of the clinical study, the number of images produced, image-related metadata (e.g., time of the image, type or manufacturer of the modality used for producing images, position of the patient for the image, identification of the facility, etc.), or the like. According to at least this example, the at least one action performed based on the results may be benchmarking or ranking facilities according to the number of patients examined and/or treated and/or the number of images delivered for the study.

Methods according to at least some example embodiments may be performed in real time. For example, data entering the study system may be automatically evaluated, a measured variable may be determined or generated and at least one action may be initiated with little, without any significant or without any time delay. All or substantially all measured variables as information about the study may be available in real time. All or substantially all actions may also be performed in real time, and thus, the study or studies may be managed more flexibly and/or instantaneously.

According to at least one example embodiment, the action may be displaying a measured variable in the form of a scorecard or a report. Such a scorecard or a report may include a brief summary of relatively (e.g., most) important information about the study. Decision makers (e.g., human operators or administrators of the study) tasked with implementing the clinical study may constantly and/or instantaneously access one or more available status reports or overviews from the study, the quality of the facilities tasked with the study, the progress of the study, etc. Using this information, incorrectly working facilities may be excluded from the study with little or no delay. A corresponding scorecard/report may be produced on request or periodically at given, specified or predefined times.

According to at least one other example embodiment, the action may be sending a message to a receiver. The receiver may be, for example, a decision maker (e.g., human operator or administrator of the clinical study) to inform the receiver of relatively important aspects of the study. A management criteria correlated to or associated with such an action may be, for example, exceeding of a threshold value for a particular measured variable. The message may be, for example, an alarm message. In one example, the message may indicate excessively rising costs during a particular phase of one or more clinical studies. The receiver, as the decision maker, may attempt to take countermeasures and/or abort the clinical study.

The ascertained measured variable may be a measured variable across one or more clinical studies. The measured variable may be relevant not only to the one or more clinical studies currently being implemented, but also to other clinical studies, for example, planned in parallel or in the future. The measured variable may be incorporated into these other clinical studies.

In another example, the action may be benchmarking or classifying the facility so as to rank it amongst other facilities. Over the course of time (e.g. after several studies have been implemented) more reliable facilities may be identified, and these facilities may be used more preferably with future studies. In another example embodiment, the action may be financial. For example, financial expense allowances for services provided in the course of the study may be implemented for one or more facilities. According to at least this example embodiment, the method may be used to compensate one or more facilities more fairly and/or based on services actually provided.

At least one other example embodiment provides a clinical study data evaluation system study management system for automatically evaluating data from at least one clinical study. The clinical study data evaluation system may include a processing unit. The processing unit may be configured to assess the data based on at least one management criteria, determine at least one measured variable based on the assessed data, and initiate at least one action correlated to a facility based on the at least one measured variable. The at least one management criteria may be associated with the at least one clinical study.
At least one other example embodiment provides a computer readable medium storing computer program instructions executable by a computer, the instructions, when executed by the computer, causing the computer to execute a method for automatically evaluating data from at least one clinical study. According to at least this method, data may be assessed at a study management system based on at least one management criteria. At least one measured variable may be determined based on the assessed data, and at least one action correlated to at least one facility may be initiated based on the at least one measured variable. The at least one management criteria may be associated with the at least one clinical study.

Although particular example embodiments are described herein, it will be understood that example embodiments may combined in a variety of ways. For example, assessments may be made based on a calculation rule, a study protocol and/or an agreement.

Example embodiments relate to all or substantially all data produced for at least one clinical study. However, as mentioned, image data determined in the course of the clinical study and metadata correlated thereto may be assessed. In the case of image-related clinical studies, example embodiments provide the ability to handle a larger volume of data as compared to conventional methods and systems.

### BRIEF DESCRIPTION OF THE DRAWINGS

The present invention will now be described with regard to example embodiments shown in the drawings, in which:

FIG. 1 is a block diagram illustrating an automatic clinical study data evaluation system according to an example embodiment.

### DETAILED DESCRIPTION OF EXAMPLE EMBODIMENTS

Various example embodiments of the present invention will now be described more fully with reference to the accompanying drawings in which some example embodiments of the invention are shown. Detailed illustrative embodiments of the present invention are disclosed herein. However, specific structural and functional details disclosed herein are merely representative for purposes of describing example embodiments of the present invention. This invention may, however, may be embodied in many alternate forms and should not be construed as limited to only the embodiments set forth herein.

Accordingly, while example embodiments are capable of various modifications and alternative forms, embodiments thereof are shown by way of example in the drawings and will herein be described in detail. It should be understood, however, that there is no intent to limit example embodiments of the invention to the particular forms disclosed, but on the contrary, example embodiments of the invention are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or” includes any and all combinations of one or more of the associated listed items.

The terminology used herein is for the purpose of describing particular embodiments only and is not intended to be limiting of example embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

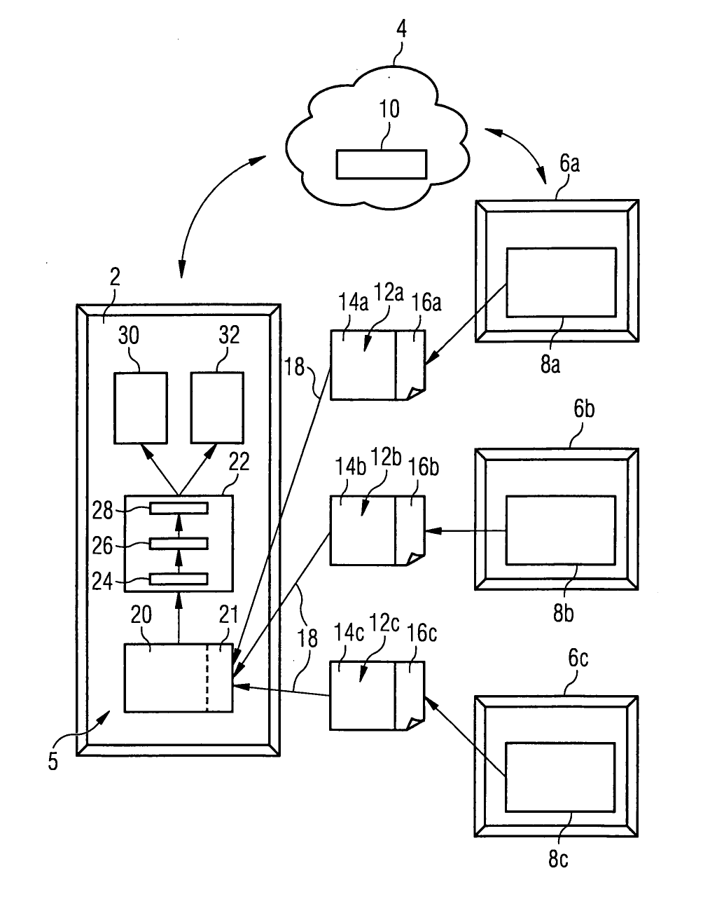

FIG. 1 is a block diagram illustrating an automatic clinical study data evaluation system according to an example embodiment. As shown, the automatic clinical study data evaluation system may include a study management system 2. The study management system may be used to implement a clinical study 4. Although only a single clinical study 4 is shown in FIG. 1, automatic clinical study data evaluation systems according to at least some example embodiments may be capable of implementing a plurality of clinical studies.

The clinical study 4 may be organized by a central station (not shown), for example, a sponsor. The clinical study 4 may involve the participation of one or a plurality of (e.g., 1, 2, 10, or more) clinics (or “sites”) at which patients are recruited and/or examined.

For example purposes, FIG. 1 shows a plurality of clinics 6a-6c. Although FIG. 1 shows three clinics, example embodiments may include any number of clinics, and should not be limited to three. A clinical study 4 may include a collection of laboratory data, clinical examinations, medical image data (e.g., magnetic resonance images (MRI), positron emission tomography (PET), or the like), etc.

The clinics 6a-6c may be contractually obligated to collect data according to one or more study protocols 10 defined by the sponsor. The study protocol 10 may stipulate, for example, times at which data (e.g., image data, etc.) is collected, the manner in which the data is collected, etc. More generally, for example, the study protocol 10 may identify methods, equipment and/or test sequences used to collect the data, such that all or substantially all data may be collected in a more uniform manner. This may allow all or substantially all collected data to be compared.

In one example, according to the study protocol 10, image data (e.g., MRI images) may be taken at designated or given times (e.g., every four weeks) over a first time period (e.g., one-half year) in parallel with a prescribed treatment (e.g., drug therapy). Each clinic 6a-6c may include an imaging modality 8a-8c, and the data captured in the clinics 6a-6c may be captured electronically using the imaging modalities 8a-8c.

In accordance with instructions provided in the study protocol 10, the clinics 6a-6c may use imaging modalities 8a-8c to examine and produce images 12a-12c of patients (not shown). In this example, the images 12a-12c contain actual image data 14a-14c and associated information 16a-16c (e.g., metadata), respectively. The image data 14a-14c may be preprocessed by the image modalities 8a-8c, respectively. The associated information 16a-16c may be associated with images 12a-12c, respectively, and may describe a MR sequence, such as the position, arrangement and/or situation of the patient, the image times (e.g., length or time the image is taken), the modality type, etc. The information 16a-16c may be stored as header data in a form representing all or substantially all additional information correlated to or associated with (e.g., directly correlated to or associated with) the image data 14a-14c, respectively. For example, the information 16a-16c may be additional information for PETS. Thus, header data for the image data 14a-14c may contain additional information about the respective image modalities 8a-8c, the clinic 6a-6c, the relevant patient and/or picture parameters (not shown), etc.

All or substantially all images 12a-12c produced in the course of the clinical study may be transmitted from the clinics 6a-6c to the study management system 2 (or central station) as indicated by arrows 18 in FIG. 1. Depending on the clinic 6a-6c, data may be transferred via a direct or indirect electronic connection (e.g., data path or data network), or using portable media, such as, a wireless network.

The study management system 2 may receive the images 12a-12c at data input module 21 in data management system 5. The data input module 21 may be part of a central support system 20. The images 12a-12c may be output from the central support system 20 to processing system 22.

Alternatively, the data input module 21 may be in standalone form. For example, the data input module 21 and/or the central support system 20 may be located apart (e.g., geographically separated) from the data management system 5. In this example, a data link may be provided between the central support system 20, the data input module 21 and the processing system 22.

According to at least some example embodiments, after each data collection (e.g., as a result of a patient visit to the clinic 6a-6c), a data record may be available to the data input module 21. The data record may be in the form of the images 12a-12c. The data record may be transmitted to the data management system 5, at which all or substantially all data (e.g., images 12a-12c) may be gathered, checked, analyzed and/or exported for analysis. In this example, the data may be transmitted to the study management system 2 in accordance with the study protocol 10.

In addition, the data input module 21 may apply algorithms to extract information relevant to the management of the clinics 6a-6c. In one example, data 24 may be extracted from both the image data 14a-14c and/or the information 16a-16c through further processing. For example, data 24 may be extracted from the Digital Imaging and Communications (DICOM) header associated with the images 12a-12c. The extracted data 24 may be compared with conditions, stipulations and/or characteristics associated with or included in the study protocol 10. The comparison may be performed using at least one rule 26. The rule 26 may be a management criteria or criterion. Using or based on the data 24 and the rule 26, a result 28 may be calculated. The result 28 may be a measured variable.

A more specific example will now be described below. In this example, the following results will be assumed to have been generated for each of clinics 6a-6c:
1. For two of five MRI pictures, clinic 6a has not observed the position of the patient as stipulated in the study protocol 10;
2. Clinic 6b has produced seventy MRI pictures, for which the patient was in the machine for X minutes on average; and
3. Clinic 6c has six different radiologists examining the images 12c, whereas clinic 6b has only a single radiologist for the same volume of image data.

The determined results 28 may be forwarded to the study management system 2 in the central station, which visually displays this information or links to other information. Based on the rule 26, presentations 30 of the results 28 may be produced and/or displayed as actions.

The study management system 2 may also initiate actions 32 based on, for example, business rules. Business rules may be derived, for example, from a contract between clinics 6a-6c and the central station. These actions 32 may be, for example, financial transactions. For example, the study management system 2 may initiate a payment to a clinic 6a-6c for a full data record associated with a patient (e.g., a participant in the study). Alternatively, a payment may be refused because the images 12a-12c do not meet the conditions of the contract. In another example, a clinic 6a-6c may be excluded from further participation in the clinical study 4 because the delivered data does not comply with the study protocol 10. All such or similar actions may be used to assist in managing of the participating clinics 6a-6c.

Methods according to example embodiments may be machine implemented via one or more computers or processors. In addition, the systems discussed herein may be embodied in the form of one or more computers configured to carry out methods described herein.

Example embodiments may also be implemented, in software, for example, as any suitable computer program. For example, a program in accordance with one or more example embodiments of the present invention may be a computer program product causing a computer to execute one or more of the example methods described herein: a method for automatically evaluating data from a clinical study.

The computer program product may include a computer-readable medium having computer program logic or code portions embodied thereon for enabling a processor of the apparatus to perform one or more functions in accordance with one or more of the example methodologies described above. The computer program logic may thus cause the processor to perform one or more of the example methodologies, or one or more functions of a given methodology described herein.

The computer-readable medium may be a built-in medium installed inside a computer main body or removable medium arranged so that it can be separated from the computer main body. Examples of the built-in medium include, but are not limited to, rewriteable non-volatile memories, such as RAMs, ROMs, flash memories, and hard disks. Examples of a removable medium may include, but are not limited to, optical storage media such as CD-ROMs and DVDs; magneto-optical storage media such as MOs; magnetism storage media such as floppy disks (trademark), cassette tapes, and removable hard disks; media with a built-in rewriteable non-volatile memory such as memory cards; and media with a built-in ROM, such as ROM cassettes.

These programs may also be provided in the form of an externally supplied propagated signal and/or a computer data signal (e.g., wireless or terrestrial) embodied in a carrier wave. The computer data signal embodying one or more instructions or functions of an example methodology may be carried on a carrier wave for transmission and/or reception by an entity that executes the instructions or functions of the example methodology. For example, the functions or instructions of the example embodiments may be implemented by processing one or more code segments of the carrier wave, for example, in a computer, where instructions or functions may be executed for automatically evaluating data from a clinical study, in accordance with example embodiments described herein.

Further, such programs, when recorded on computer-readable storage media, may be readily stored and distributed. The storage medium, as it is read by a computer, may enable the automatic evaluating of data from a clinical study in accordance with the example embodiments described herein.

Example embodiments being thus described, it will be obvious that the same may be varied in many ways. For example, the methods according to example embodiments of the present invention may be implemented in hardware and/or software. The hardware/software implementations may include a combination of processor(s) and article(s) of manufacture. The article(s) of manufacture may further include storage media and executable computer program(s), for example, a computer program product stored on a computer readable medium.

The executable computer program(s) may include the instructions to perform the described operations or functions. The computer executable program(s) may also be provided as part of externally supplied propagated signal(s). Such variations are not to be regarded as departure from the spirit and scope of the example embodiments, and all such modifications as would be obvious to one skilled in the art are intended to be included within the scope of the following claims.

The present invention being thus described, it will be obvious that the same may be varied in many ways. Such variations are not to be regarded as a departure from the invention, and all such modifications are intended to be included within the scope of the present invention.

We claim:

1. A method for automatically evaluating data from at least one clinical study, the method comprising:
assessing, at a study management system, the data based on at least one management criteria, the at least one management criteria being associated with the at least one clinical study;
determining at least one measured variable based on the assessed data; and
initiating at least one action correlated to at least one facility based on the at least one measured variable.

2. The method of claim 1, further comprising:
supplying the data from a data management system to the study management system via at least one data path.

3. The method of claim 1, wherein the at least one management criteria includes at least one of a study protocol and an agreement with the at least one facility, the agreement being associated with the clinical study.

4. The method of claim 1, wherein the at least one measured variable is determined using at least one calculation rule associated with the clinical study.

5. The method of claim 1, wherein the data includes at least one of image data and metadata correlated to the image data.

6. The method of claim 1, wherein the assessing, determining and initiating are performed in real time.

7. The method of claim 1, wherein the at least one action is displaying the at least one measured variable in the form of a scorecard or a report.

8. The method of claim 1, wherein the at least one action is sending at least one message to a receiver associated with the at least one facility.

9. The method of claim 1, wherein the at least one measured variable is measured across the at least one clinical study.

10. The method of claim 1, wherein the at least one action is benchmarking the at least one facility.

11. The method of claim 1, wherein the at least one action is implementing a financial expense allowance for the at least one facility, the financial expense allowance being associated with services provided in the course of the at least one clinical study.

12. A study management system for automatically evaluating data from at least one clinical study, the system comprising:
a processing unit configured to,
assess the data based on at least one management criteria, the at least one management criteria being associated with the at least one clinical study,
determine at least one measured variable based on the assessed data, and
initiate at least one action correlated to a facility based on the at least one measured variable.

13. The study management system of claim 12, further comprising:
a data input module configured to receive the data, and output the data to the processing unit.

14. The study management system of claim 13, wherein the data input module is separate from the study management system, and the study management system further comprises:
a data path for transmitting the data from the data input module to the study management system.

15. The study management system of claim 13, wherein the data input module is integrated into the study management system.

16. The study management system of claim 12, wherein the at least one management criteria includes at least one of a study protocol and an agreement with the facility concerning the study.

17. The study management system of claim 12, wherein the at least one measured variable is determined using at least one calculation rule associated with the at least one clinical study.

18. The study management system of claim 12, wherein the data includes at least one of image data and metadata correlated to the image data.

19. The study management system of claim 12, wherein the at least one action includes at least one of displaying the at least one measured variable in the form of a scorecard or a report, sending at least one message to a receiver associated with the at least one facility, and benchmarking the at least one facility.

20. The study management system of claim 12, wherein the at least one action includes implementing a financial expense allowance for the at least one facility, the financial expense allowance being associated with services provided in the course of the at least one clinical study.

21. The study management system of claim 12, wherein the at least one measured variable is measured across the at least one clinical study.

22. A computer readable medium storing computer program instructions executable by a computer, the instructions, when executed by the computer, causing the computer to execute a method for automatically evaluating data from at least one clinical study, the method comprising:
assessing, at a study management system, the data based on at least one management criteria, the at least one management criteria being associated with the at least one clinical study;
determining at least one measured variable based on the assessed data; and
initiating at least one action correlated to at least one facility based on the at least one measured variable.

23. The computer readable medium of claim 22, wherein the at least one management criteria includes at least one of a study protocol and an agreement with the at least one facility concerning the at least one clinical study.

24. The computer readable medium of claim 22, wherein the at least one measured variable is determined as using at least one calculation rule associated with the at least one clinical study.

25. The computer readable medium of claim 22, wherein the data includes at least one of image data and metadata correlated to the image data.

26. The computer readable medium of claim 22, wherein the assessing, determining and initiating are performed in real time.

27. The computer readable medium of claim 22, wherein the at least one action is displaying the at least one measured variable in the form of a scorecard or a report.

28. The computer readable medium of claim 22, wherein the at least one action is sending a message to a receiver associated with the at least one facility.

29. The computer readable medium of claim 22, wherein the at least one measured variable is measured across the at least one clinical study.

30. The computer readable medium of claim 22, wherein the at least one action is benchmarking the at least one facility.

31. The computer readable medium of claim 22, wherein the at least one action is implementing a financial expense allowance for the at least one facility, the financial expense allowance being associated with services provided in the course of the at least one clinical study.
